(19) United States
(12) Patent Application Publication
(10) Pub. No.: US 2009/0053197 A1
(43) Pub. Date: Feb. 26, 2009

(54) Transfer Factor Compositions and Methods

(76) Inventor: Joseph C. Ramaekers, Aptos, CA (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 11/762,727

(22) Filed: Jun. 13, 2007

Related U.S. Application Data
(60) Provisional application No. 60/814,777, filed on Jun. 14, 2006.
(60) Provisional application No. 60/834,739, filed on Jul. 31, 2006.

(51) Int. Cl. A61K 39/395; A61P 3/00

(52) U.S. Cl. 424/130.1

(57) ABSTRACT

Compositions are provided comprising transfer factor alone or combined with an antibody. The antibody may be contained in an antibody fraction. The transfer factor and/or the antibody or antibody fraction may be lyophilized. Also provided are formulations further comprising glucans, as well as additional optional components. Also provided are methods for making the compositions and formulations, as well as kits containing the compositions. Methods of preventing and/or treating a condition in a subject using the compositions and/or formulations are also provided. Such conditions may include malignant and benign tumors.

### CROSS-REFERENCE TO RELATED APPLICATIONS

This application claims benefit of U.S. Provisional Application No. 60/814,777, filed Jun. 14, 2006, and also claims benefit of U.S. Provisional Application No. 60/834,739, filed Jul. 31, 2006, the disclosures of which are hereby incorporated by reference herein, in their entireties.

### FIELD OF THE INVENTION

This invention relates generally to compositions comprising transfer factor, particularly lyophilized transfer factor; and to compositions comprising transfer factor in combination with an antibody. Such compositions are useful in the prevention and/or treatment of certain conditions, including benign and malignant tumors.

### BACKGROUND OF THE INVENTION

Transfer factors, which are produced by leucocytes and lymphocytes, are small water soluble polypeptides of between about 44 amino acids that stimulate or transfer cell mediated immunity from one individual to another and across species but do not create an allergic response. Since transfer factors are smaller than antibodies, they do not transfer antibody mediated responses nor do they induce antibody production. The properties, characteristics and processes for obtaining transfer factor or transfer factors are discussed in U.S. Pat. Nos. 4,816,563; 5,080,895; 5,840,700, 5,883,224 and 6,468,534, the contents of which are hereby incorporated by reference into the present application.

Transfer factor has been described as an effective therapeutic for Herpes simplex virus (Viza, et al.), a treatment for acne blemishes, U.S. Pat. No. 4,435,384 and as a treatment against C. albicans (Khan et al.). Transfer factor has also been used to treat intestinal cryptosporidiosis in recipients treated with specific transfer factor (McMeeking, et al.). Still, et al. also showed that chicken pox infections were prevented by pretreatment of children treated with transfer factor from individuals that had chicken pox or who in other words had been sensitized to the varicella antigen. The antigen specific transfer factors are the most well studied and have been demonstrated to be able to convey the antigen recognition ability of the experienced donor to the naive recipient. It may be assumed that the individual or animal that is the source of the transfer factor has been sensitized to the antigen of interest. However, transfer factor as found in commercial bovine colostrum extract coming from a pool of animals (e.g., cows) contains the acquired immunity from all of the pool and therefore provides a type of generalized adoptive transfer of immunity. Transfer factors or transfer factor can be obtained from a dialyzable extract of the lyzed cells or from an extract of extracellular fluid containing transfer factor. Common sources of transfer factors are colostrums and ova. It is common practice to refer to preparations that contain transfer factor by the name of the active component (i.e., transfer factor or TF). Transfer factor extract containing transfer factors is also herein referred to as transfer factor. Transfer factor from bovine colostrum extract is defined as defatted water soluble material from colostrum that will pass through a nominal 10,000 molecular weight filter. The colostral derived transfer factor has been prepared with activity against various organisms including infectious bovine rhinotracheitis virus. One of the specific effects of transfer factor is a significantly increased natural killer (NK) cell activity. Natural killer cells provide protection against viruses as part of the innate immune defense system.

Although transfer factor is a polypeptide, it has been reported that it is surprising stable in the gastrointestinal tract. For example, Kirkpatrick compared oral versus parental administration of transfer factor in clinical studies. Kirkpatrick, Biotherapy, 9:13-16, 1996. He concluded that the results refute any arguments that the acidic or enzymatic environment of the gastrointestinal tract would prevent oral therapy using transfer factors.

When attempts were made to sequence TF, it was reported that an N-terminal end of the transfer factor peptide is resistant to sequential Edman degradation. Kirkpatrick, Molecular Medicine, 6(4):332-341 (2000).

Accordingly, transfer factor was believed to be stable in the gastrointestinal tract and rumen. However, it has since been shown that transfer factor is not as stable as once believed. It appears to be particularly unstable in the digestive tract of ruminants.

Transfer factors have been used successfully in compositions for treating animal diseases and syndromes including those in ruminants. See, for example U.S. Pat. No. 6,962,718.

The present invention relates to compositions and formulations containing transfer factor, as well as methods of making the same and methods of treatment and/or prevention of conditions using the same. Other U.S. patents and U.S. patent applications relate to the present invention, including without limitation, U.S. Pat. Nos. 6,506,413 and 6,962,718, U.S. Patent Provisional Application Nos. 60/573,113, 60/649,363, 60/701,860, and 60/814,777, U.S. Patent Application Publication Nos. 2006/0029585 A1, 2006/0073197 A1, and 2007/0128253 A1, all of which are incorporated herein by reference. Also related are PCT publications WO/2002/087599 and WO/2005/112891, incorporated herein by reference.

### BRIEF SUMMARY OF THE INVENTION

In certain aspects, the invention relates to compositions comprising lyophilized transfer factor. In other aspects, the invention relates to compositions comprising transfer factor in combination with an antibody. In certain aspects, the antibody is contained in an antibody fraction. In certain embodiments, the transfer factor and/or the antibody or antibody fraction with which it is combined may be lyophilized.

In further aspects, compositions and formulations of the invention may further comprise additional components. In certain preferred embodiments, the compositions and formulations comprising transfer factor and/or antibody or antibody fraction may additionally comprise glucans.

Additional aspects of the invention relate to compositions and formulations comprising encapsulated components; including, but not limited to, encapsulated transfer factor and/or encapsulated antibody or antibody fraction.

Additional aspects of the present invention are directed to methods of making compositions and formulations according to the invention.

Further aspects of the present invention are directed to methods of treating and/or preventing certain conditions comprising administering an effective amount of a composition and/or formulation comprising transfer factor and/or antibody or antibody fraction.

Both the foregoing general description and the following detailed description are exemplary and explanatory, and are intended to provide further explanation of the invention as claimed. Other objects, advantages, and novel features will be readily apparent to those skilled in the art from the following detailed description of the invention.

### BRIEF DESCRIPTION OF THE FIGURES

FIG. 1 shows the results of an assay for lymphocyte stimulation following application of a formulation containing lyophilized transfer factor.

FIG. 2 shows the results of an assay for lymphocyte stimulation following application of a formulation containing glucans.

### DETAILED DESCRIPTION OF THE INVENTION

In certain embodiments, the present invention is directed to compositions comprising transfer factor and an antibody. In certain embodiments, the antibody is contained within an antibody fraction.

In other embodiments, the invention is directed to compositions comprising transfer factor that is lyophilized. In certain embodiments, lyophilized transfer factor may be combined with antibody or antibody fraction. In certain preferred embodiments of the invention, compositions are provided comprising lyophilized transfer factor and lyophilized antibody. In certain embodiments, the antibody may be in an antibody fraction that is lyophilized.

Other aspects of the invention are directed to formulations further comprising components, including, but not limited to, nutraceutical ingredients, in addition to lyophilized transfer factor. In other aspects of the invention, there are provided formulations comprising additional components in addition to transfer factor and antibody or antibody fraction.

Transfer Factor

According to particular embodiments of the invention, compositions and formulations are provided comprising transfer factor. According to certain embodiments of the invention, various forms of transfer factor may be used. They include, without limitation, excreted transfer factor released from transfer factor containing cells such as lymphocytes, leukocytes, and ova, and collected from extracellular fluids such as colostrums and blood. Another form includes preexcreted transfer factor found within the cell or on the cell surface. In certain embodiments of the invention, substantially purified transfer factor originating from leukocytes, colostrum, or ova and having a molecular weight of less than 10,000 daltons and a specific activity of at least 5000 units per absorbance unit at 214 nanometers, may also be used. The transfer factor used in the Examples of this invention and referred to in the following Tables and further referred to in the rest of the detailed description is generally extracted from colostrum collected from a general pool of lactating cows; although, in some cases, it is derived from eggs. Though bovine colostral derived transfer factor was generally used to develop the formulations of this invention, it is well known to anyone skilled in the art that other kinds and sources of transfer factor could be used.

Alternative sources of transfer factor include, but are not limited to, avian transfer factor, ova transfer factor, and transfer factor isolated from colostrum collected from non-bovine animals such as goats, pigs, horses and humans. In addition, combinations of transfer factors from any number of sources may be used in the formulations of the instant invention. Transfer factor may also be derived from recombinant cells that are genetically engineered to express one or more transfer factors or by clonal expansion of leukocytes.

Isolation of Transfer Factor and Antibody Fraction

In certain embodiments of the invention, transfer factor may be obtained from colostrum. In a preferred embodiment, transfer factor is obtained from bovine colostrum. The colostrum is fractionated, removing most of the curds and whey, to produce the transfer factor and antibody fractions. The fraction having a molecular weight of approximately 10,000 daltons (Da) or below is designated as transfer factor. The fraction obtained that is approximately 10,000 to 150,000 Da is designated the antibody fraction, also known as the antibody-colostrum fraction. In certain embodiments, the antibody fraction comprises antibodies from about 1% to about 99%, about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, or about 50% by weight, the remainder comprising other colostrum components.

According to certain embodiments of the invention, transfer factor, as used in the formulations described in the Tables, particularly when not defined as obtained from an avian source, may be further defined as defatted water soluble material from bovine colostrum that will pass through a nominal 10,000 molecular weight filter.

In other embodiments, the transfer factor is obtained from an avian source. In one embodiment, chickens are given a feed mixture containing excrement from an animal, including without limitation, a human, a fish, a goat, a llama, an alpaca, a pig, a sheep, a cow, and a horse. The excrement will contain a large variety of pathogens and upon administration of a feed to an animal, it will develop transfer factor and/or antibodies to such pathogens. Avian transfer factor can then be obtained from the eggs produced by the above-treated chickens. In certain embodiments of the invention, transfer factor may be found in whole egg yolks. As a non-limiting example, the transfer factor of avian source (which is believed to also contain antibodies) listed in the formulation of Table 7 is supplied as powdered whole egg yolks.

Alternative kinds of transfer factor include, but are not limited to, targeted transfer factors. Target transfer factors include transfer factor collected from sources which have been exposed to (1) one or more viral or otherwise infectious organisms; (2) one or more antigens that produce an immune response; or (3) a combination of organisms and antigens. The term antigen is defined herein is anything that will initiate the cell mediated immune response. Examples of such viral or other infectious organisms include Herpes Simplex Virus 1, Herpes Simplex Virus 2, H. Pylori, Camphobactor and Chlamydia, Bovine Rhinotracheitis Virus, Parainfluenza, Respiratory Syncytial Virus Vaccine, modified live virus, Campylobacter Fetus, Leptospira Canicola, Grippotyphosa, Hardjo, Leterohaemorrhagiae, Pomona Bacterin, Bovine Rota-Coronavirus, Escherichia Coli Bacterin, Clostridium Chauvoei, Septicum, Haemolyticum, Novy, Sordellii, Perfringens Types C & D, Bacterin, Toxoid, Haemophilus Somnus, Pasteurella Haemolytica, Multocida Bacterin. However, one of skill in the art would readily recognize that a wide variety of other viral and otherwise infectious organisms can find use in the instant invention. Examples include those set forth in Appendix I and Appendix II.

Antibodies

In another aspect of the present invention, the formulations and compositions of the present invention include an antibody. The antibody may be present in an antibody fraction. In certain embodiments, the antibody or the antibody fraction is present in a composition also comprising transfer factor. The antibody or antibody fraction may be present at about 1% to about 99% of a composition having the transfer factor. In other embodiments, the antibody or antibody fraction is present from about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, or about 50% of a composition having the transfer factor. In other embodiments, the antibody or antibody fraction is present at about 15% to about 25%, about 17.5% to about 22.5%, or about 20% of a composition having a transfer factor. In some embodiments, the antibody is provided as a lyophilized antibody or antibody fraction.

Lyophilization

The present invention also provides compositions, formulations, and kits containing the same, that have one or more lyophilized component(s). Lyophilization or “freeze-drying” is a process well known to those of ordinary skill in the art. For example, some techniques of lyophilization are described in Akers, Michael J., Chapter 41 in Remington The Science and Practice of Pharmacy, 828 (David B. Troy ed., Lippincott Williams & Wilkins 2006), which is incorporated herein by reference. In certain embodiments, formulations and/or compositions of the present invention may include lyophilized transfer factor. In certain embodiments, transfer factor, which may be lyophilized, may be combined with antibody or an antibody fraction, which may, in certain embodiments, be lyophilized. In other embodiments, additional components of formulations and compositions of the invention may be lyophilized, including, without limitation, other peptides and proteins.

In certain embodiments, the transfer factor is present in a composition also comprising antibody or antibody fraction. In certain embodiments, the transfer factor is present from 1% to about 99% by weight in a composition also comprising antibody or antibody fraction. In other embodiments, the transfer factor is present from about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, or about 50%, all by weight of a composition also comprising antibody or antibody fraction. In certain embodiments, transfer factor is present in a composition from about 70% to about 90%, about 75% to about 85%, or about 77.5 to about 82.5% by weight of a composition comprising antibody or antibody fraction. In certain preferred embodiments, the transfer factor is present in a composition at approximately 80% by weight of a composition also comprising antibody or antibody fraction.

### Formulations

In certain embodiments of the invention, transfer factor is provided in a formulation that further comprises one or more additional ingredients. In some embodiments, transfer factor and an antibody or antibody fraction is provided in a formulation that further comprises one or more additional ingredients. In certain embodiments, the transfer factor may be lyophilized. In certain embodiments, the antibody or antibody fraction may be lyophilized.

In a preferred embodiment, transfer factor is present in the formulation in the amount of about 10 mg to about 12 gm/oz, more preferably about 100 mg to about 6 gm/oz and most preferably about 10 mg to about 3 gm/oz. In certain preferred embodiments, such a formulation comprising transfer factor is provided to an animal in an amount of about 1 oz per 1000 lb of animal.

In certain embodiments of the invention, formulations are provided which comprise glucans. Glucans may be derived from any suitable source, including, but not limited to, fungi, oats, and yeast. Preferably, glucans are present in or derived from fungi.

In certain embodiments, the glucans which may be included in the formulations are present in whole fungi.

In certain preferred embodiments, glucans are present in or derived from Cordyceps, more preferably, Cordyceps sinensis.

In certain embodiments, glucans are derived from hybrid strains of fungi. In a preferred embodiment the hybrid glucans used in the invention are present in, or derived from, hybrid strains of Cordyceps and in particular Cordyceps sinensis.

One technique to induce the hybridization of Cordyceps involves plating two different strains or species on a single agar plate which has been inoculated with rattlesnake venom as described in, for example, U.S. Patent Application Publication No. 2006/0073197, published Apr. 6, 2006, and U.S. Patent Application Publication No. 2007/0128253, published Jun. 7, 2007, each of which is incorporated herein by reference. In a preferred embodiment, the hybrid strain producing the hybrid glucans that may be used in compositions and formulations of the invention is Cordyceps sinensis Alohaensis, which is available from Pacific Myco Products, Santa Cruz, Calif.

There are a number of different Cordyceps sinensis strains and due to their variable asexual mycelial growth forms they have been considered to be different species by many taxonomists. A non-exhaustive list of strains includes: Paecilomyces hepiali Chen, Cephalsporim sinensis, Paecilomyces sinensis Cn80-2, Scydalilum sp., Hirstutella sinenis, Mortierella hepiali, Chen Lu, Topycladium sinensis, Scytalidium hepiali, G. L. Li. Preferred embodiments of the instant invention make use of hybrid glucans from hybrids of one or more of these different strains, however, the invention may alternatively preferentially include glucans from non-hybridized strains. Alternative embodiments utilize the whole hybrid Cordyceps, e.g., Cordyceps sinensis Alohaensis. Hybrid glucans may also include those obtained by crossing sources of feed, e.g., oats, etc.

When glucans are used, the formulation preferably contains about 10 mg to about 18 gm of whole organism/oz, more preferably about 100 mg to about 10 gm of whole organism/oz and most preferably about 100 mg to about 5 gm of whole organism/oz.

Equivalent amounts of purified or partially purified glucan as well as the nucleosides associated therewith (e.g., Cordycepin (3′deoxyadenosine), adenosine and N6-(2 hydroxyethyl)-adenosine) may also be used.

In certain embodiments, compositions and formulations comprising transfer factor may be combined with minerals, antioxidants, amino acids, and other neutraceuticals.

The use of nutraceuticals to treat vitamin and mineral deficiencies is well known. However, the use of nutraceuticals, such as vitamins, minerals and other nutritional components to prevent and treat diseases other than those caused by the deficiency of those nutraceuticals, though still controversial, is receiving more consideration from both laymen and physicians. The following is a non-limiting list of nutraceuticals and some of their generally acknowledged nutritional and health benefits. Any of these may be included in formulations comprising transfer factor, including lyophilized transfer factor and/or transfer factor in combination with an antibody or an antibody fraction.

Vitamin A—is important in preventing eye epithelial disorders; deficiency results in night blindness

Vitamin B2—is essential to human nutrition relating to the oxidation of carbohydrates and amino acids

Mixed tocopherols—are antioxidants

Choline Chloride—is a member of the vitamin B complex and a dietetic factor for furnishing free methyl groups for transmethylation.

Vitamin B6—functions in the formation and breakdown of amino acids and is involved in the synthesis of serotonin and norepinephrine. However, exact dietary requirements are uncertain

Vitamin B12—is an antipernicious-anemia factor essential for normal hemopoiesis.

Vitamin E—is an antioxidant that protects against free radicals.

Vitamin K—is essential for the formation of prothrombin

Biotin—functions in metobolic processes leading to the formation of fats and utilization of carbon dioxide

Folic Acid—a growth factor involved in the formation of nucleic acids and necessary for the formation of heme

Niacin—a component of the Vitamin B complex, a deficiency results in pellagra

Vitamin D3—is important in the absorption of calcium

Pantothenic Acid—is considered essential for growth and well being of animals; deficiency results in growth retardation, skin lesions and graying of hair

Thiamine—is necessary in diet of all animals except ruminants; used to prevent beriberi and important in carbohydrate metabolism

Lysine—is an essential amino acid

Methionine—is a sulfur containing essential amino acid

Arginine—is an amino acid important in the synthesis of urea (principal form in which mammals excrete)

Soy—is a source of proteins

Methyl Sulfonyl Methane—is a form of organic sulfur involved in cell membrane permeability

Zinc—is an essential mineral for growth; deficiency creates susceptibility to various pathogens

Omega 3-, 6-, and 9-Fatty Acids—are essential fatty acids and polyunsaturated fats; a deficiency results in hypertension and high blood pressure; they are believed to improve immune function

Yeast—(e.g., brewers, bakers, etc.) contains beta glucans which appear to increase production and/or activation of natural killer cells

Calcium—is required for bone development

Phosphorus—is required for bone development

Selenium—a deficiency results in heart muscle disease

Iron—is required for formation of hemoglobin; deficiency results in anemia

Magnesium—is an element required for growth in all living organisms

Manganese—is an element required for growth in all living organisms

Copper—is an element required for growth in plants, animals and most microorganisms

Iodine—is an element necessary for the synthesis of hormone production by the thyroid gland

Cobalt—is a trace element essential in the nutrition of ruminants (cattle, sheep) and in the maturation of human red blood cells in the form of Vitamin B.sub. 12

Molybdenum—is a trace element believed to be necessary in animal diets but its function in the minimal levels have not been established

Lactic Acid Generating Bacteria—are a digestive aid and growth inhibitor of harmful bacteria

Chrondroitin—is a component of connective tissue which may relieve joint pain and arthritis.

Glucosamine—is a component of micropolysaccharides and glycoprotein which may be helpful in arthritis.

Di-methyl glycine—is a methylated amino acid found in all cells and an antioxidant.

Montmorillonite—is collodial clay containing trace elements which are considered by some to be important for well being and to compensate for elements no longer in foods because of depleted soils (the components are shown below in Table 1)

Super oxide dismutase (SOD)—is an antioxidant enzyme present in the mammalian body. It converts super oxide free radicals to the less active peroxide. It stimulates hair growth and is believed to protect cells against ultraviolet-B irradiation and to protect the heart.

Boswellia—is an herb Boswellia serrata. Boswellic acids, the biologically active ingredients of the gum resin of this herb, are considered to have anti-inflammatory and anti-arthritic actions.

Octocosonol—is derived from wheat germ oil and provides 17% more residual energy before fatigue.

In certain preferred embodiments of the invention, formulations useful for the prevention and/or treatment of conditions in a subject, such as, for example, a mammal, may include one or more of the following: lyophilized transfer factor (mammalian) in combination with mammalian antibody-colostrum fraction, avian antibodies or antibody fraction (may, in certain embodiments, be obtained from whole egg yolk), glucans, preferably hybrid glucans, essential fats, lactic acid producing bacteria, Vitamin C, zinc, 1p6 (Inositol hexaphosphate), ace mannins, olive leaf extract, phytosterols, montmorillinite, amino acids, Methyl Sulfonyl Methane, and choline bitartrate, as well as additional vitamins and minerals.

In other preferred embodiments of the invention, formulations may additionally comprise one or more of glucosamines, chondroitins, Boswella, tumeric, and super oxide dismutase. In certain embodiments, adding one or more of these ingredients may make the formulation particularly effective in treating cancer, as pain reduction and cutting inflammation appear to be a large factor in cancer remission, as well as getting the animal to eat.

Table 1 sets forth typical components of Montmorillonite.

Tables 2-6 set forth transfer factor formulations that have been used to treat various animals and pathologies. In each case, the transfer factor is not lyophilized as set forth herein. However, the transfer factor in each of these formulations can be readily lyophilized prior to admixture with the other components of the formulation. In certain embodiments, transfer factor may be added to these formulations along with antibody or an antibody fraction. In such embodiments, the transfer factor and/or the antibody or antibody fraction may be lyophilized.

Table 2, shows a breakdown of a formulation of transfer factor, nutraceuticals and carriers useful for treating a number of conditions, including, without limitation, Cushing syndrome, Cushings disease, adenomas, onchocerciasis, hypothyroidism or EPM. In Table 2 and all the other tables references to “lb” (pounds) means pounds of body weight.

Columns 2, 3 and 4 of Tables 2-6 show the approximate high, low and preferred amounts, respectively, of the formulation components, in amounts per body weight, to be given to an animal in a single dosage. The formulations in Tables 3 and 4 are very similar to the formulation of Table 2 but they are preferably used for dogs and cats, respectively. The formulation represented in Table 2 is designed preferably for livestock. The 5 ounces of the formula listed in column 5 is designed to be given to a 1000 pound animal but that will vary and could be given to a 500 pound animal in some cases. The average horse is around 1000 pounds. The 28.3 gm dosage in Table 3 is calculated for a dog weighing about 100-200 pounds but that dosage may also be given to a 15 pound dog. The 2.2 gm formula in Table 4 is for a cat weighing around 15 pounds. However, since these formulas are comprised of nutraceuticals and transfer factor, one skilled in the art will recognize that the ranges are not certain and as critical as the ranges for allopathic drugs.

Further, the formulations in Tables 2-4 are designed to treat preferably chronic diseases, the formulation in Table 5 is designed for treatment preferably of acute diseases and the formulation in Table 6 is useful for both acute and chronic diseases. All the formulations may be given in megadoses to achieve an acute response.

In certain embodiments, the invention provides compositions in which a transfer factor and/or antibody or antibody fraction is “encapsulated.” The encapsulation protects the transfer factor and/or antibody or antibody fraction from inactivation in the gastrointestinal tract. Such encapsulation is important especially in the case of ruminants where digestion within the rumen has been found to be problematic. Enhanced bioavailability has been demonstrated when a transfer factor is encapsulated and administered to ruminants. In preferred embodiments, the transfer factor and/or antibody or antibody fraction is encapsulated by mixing with a hydrophobic substance or a lipid to form a coating around the transfer factor and/or the antibody or antibody fraction. In certain aspects, the composition may contain encapsulated glucans. Other optional components of compositions and formulations of the invention may be encapsulated, such as, without limitation, β-sitosterol, inositol hexaphosphate, olive leaf extract, aloe extract, vitamin C, and glucans, including, but not limited to, glucans obtained from fungi as described herein. The transfer factor and/or antibody or antibody fraction can be individually encapsulated or encapsulated as a mixture. Alternatively, the entire formulation can be encapsulated. The encapsulated transfer factor and/or encapsulated antibody formulation can be produced in a variety of ways. In a preferred embodiment, each of the transfer factor and/or antibody or antibody fraction in the formulation is encapsulated as described in U.S. Pat. Nos. 5,190,775, 6,013,286 and U.S. Application 2003/0129295, each of which is incorporated herein by reference in their entirety.

In certain embodiments, glucans of the formulation may be encapsulated, preferably with a hydrophobic or lipid coating. It is preferred that the amount of hydrophobic or lipid coating be between about 25% and 150 wt % of the glucan, about 50-150 wt %, or about 75-125 wt % with an equal weight being most preferred.

Table 7 provides an encapsulated transfer factor formulation for treating pathologies. This transfer factor formulation includes at least encapsulated transfer factor derived from both bovine and avian sources, and/or one or more of hybrid glucans. It is preferred that the glucan portion of this formulation also be encapsulated. Other components include zinc proteinate, targeted avian transfer factors, β-sitosterol, inositol hexaphosphate (IP6), olive leaf extract, aloe extract powder, probiotics, B. subtlis, B. longum, B. thermophilium, L. acidophilus, E. faecium, and S. cerevisia. In a preferred embodiment, all of the foregoing are included in this transfer factor formulation.

In another preferred embodiment, a formulation is provided according to Table 7, but with the following modifications. The component listed as “Transfer factor (mammal source)” is substituted with a composition containing 80% bovine colostrum transfer factor as described herein, combined with 20% bovine colostrum antibody fraction as described herein (both weight percents of the composition). The mammalian transfer factor and the colostrum antibody fraction are both lyophilized. In addition, the component listed as “Transfer factor (avian source)” is present in the formulation in an amount of 3000.0 mg/oz. This component is supplied as powdered whole egg yolk that was obtained from hyperimmunized chickens, i.e., chickens that had been exposed to pathogens prior to laying the eggs which serve as a source of transfer factor. The avian transfer factor may be obtained from commercial sources such as, for example, 4Life® Research; Labelle, Inc., Bellingham, Wash.; Troue; and Ghen Corporation, Japan.

The transfer factor may be encapsulated with a hydrophobic or lipid coating that is preferably between about 25% and about 150 wt % of the transfer factor, about 50-150 wt % and about 75-125 wt %, with an equal weight being most preferred.

In additional embodiments of the invention, additional components may be used in the formulation. For example, IP6, β-sitosterol, olive leaf extract, aloe extract matter and/or vitamin C may be used. In preferred embodiments, IP6 is present at between 10 mg and 3 gm/oz, or one preferably between 100 mg and 2 gm/oz, and most preferably between 100 mg and 1 gm/oz. The β-sitosterol is preferable in the amount of between 10 mg and 3 gm/oz, or preferably between 100 mg and 2 gm/oz, and most preferably between 100 mg and 1 gm/oz. Olive leaf extract is preferably present in the amount of 2 mg to 2 gm/oz, more preferably between 5 mg and 1 gm/oz, and most preferably between 5 mg and 500 gm/oz. Aloe extract is preferably present at between 2 mg and 1000 mg, more preferably between 5 and 500 mg/oz, and most preferably between 5 and 250 mg/oz. Vitamin C may be present at between 10 mg/oz and 10 gm/oz, or preferably between 100 mg and 8 gm/oz, and most preferably between 100 mg and 5 gm/oz.

The amount of transfer factor and/or antibody or antibody fraction used in the formulation or the amount of formulation administered will vary depending upon the severity of the clinical manifestations presented. In addition, the amount of transfer factor administered to a recipient will vary depending upon the species from the transfer factor is derived as compared to the species of the recipient. It has been observed that transfer factor derived from bovine species administered to cattle is more efficacious than transfer factor from another species such as avian species. Accordingly, when the source of the transfer factor and recipient are different species, it is preferred that the amount of transfer factor be increased.

Administration of a formulation of a transfer factor with zinc and at least one essential fatty acid is expected to result in at least a partially effective treatment of Cushings syndrome, Cushings disease, adenomas and other benign tumors, onchocerciasis, hypothyroidism or EPM. The treatment is more effective as other nutraceuticals listed in Table 2 are added. The dosage is in milligrams per pound unless otherwise stated. The amounts of the components present in a 5 ounce transfer factor formulation containing the other preferred nutraceuticals is shown in column 5 of Table 2.

Transfer factor at a dosage of about 0.75 mg/lb transfer factor in combination with about 0.49 mg/lb zinc and 20.57 mg/lb of canola oil, safflower oil or flax oil, sources of essential fatty acids (i.e., 3, 6, 9 omega fatty acids), given once daily to an animal suffering from Cushings syndrome, Cushings disease, adenomas or other benign tumors, onchocerciasis, hypothyroidism or equine protozoal myelytis should result in approximately a 30% to 50% reduction in the size of the benign tumors and/or the symptoms of these listed diseases. All of these components should of course be pharmaceutically acceptable to the animal receiving them.

A combination of Vitamin C at about 2.16 mg/lb and 2.29 mg/lb of yeast in combination with the above listed transfer factor and other fatty acid nutraceuticals should result in approximately a 40% to 50% reduction in the size of benign tumors and/or symptoms of the above listed diseases.

It is preferred in formulations of the invention that the metal nutraceuticals are proteinated because these forms are easier for the animal to digest and also because the proteinate forms are more stable to pH. The nutraceutical components in the formulations in Tables 2-7 are the active components for treating the various described diseases and syndromes. The fillers and carriers are included to make the formulations more palatable to the animal and also to help preserve the mixture. These include silicon dioxide, maltodextrin, soy and peanut flour, peanut oil, dextrose, whey, spices and flavorings. Mixed tocopherols and choline chloride are nutraceuticals but the effective results described herein can still be achieved by deleting these two components from the formulations.

Previous use of non-encapsulated transfer factor in ruminants, e.g., cows, produced significant beneficial results. See, e.g. U.S. Patent Publication 2003/0077254, published Apr. 24, 2003 incorporated herein by reference in its entirety. Subsequently, it was discovered that transfer factor was not stable by oral administration in a stressed population of cattle. After discovering that transfer factor is inactivated in vitro in the presence of rumen fluid and flora, it was determined that prior success with transfer factor in ruminants was due to the presence of the esophageal groove. When not stressed, the esophageal groove provides partial bypass of the rumen. However, in a stressed population the esophageal groove closes and shunts the transfer factor formulation into the rumen. It was discovered that encapsulating transfer factor and/or glucans with a hydrophobic substance or a lipid to form an encapsulated formulation is sufficient to provide substantial by-pass of (e.g., 85%) of the rumen even in a stressed population.

A variety of other methods for rumen by-pass are known. In one embodiment, the encapsulated or non-encapsulated formulation is directly injected (subcutaneously, intramuscularly, or intravenously) to by-pass not only the rumen but also the entire digestive system. Similarly, intravaginal, intrarectal or other direct administration to mucus membranes, such as the eye subconjunctival, by-pass the digestive system and the rumen in particular. Alternatively, the formulation can be mixed with various solvents which allow for direct skin absorption. Furthermore, methods are known in the art to stimulate opening of the esophageal groove in various ruminants and such opening allows for immediate passage of an orally administered formulation to the gastrointestinal tract, by-passing the rumen.

Preferred embodiments for human consumption include, but are not limited to incorporation of transfer factor formulations in processed foods such as cereals, snacks, chips, or bars. Preferred embodiments for animal consumption include, but are not limited to, transfer factor formulations admixed in feed pellets, salt licks, molasses licks or other processed feed products.

In certain embodiments, the transfer factor formulations find use in increasing food conversion efficiency. Food conversion efficiency is the rate at which an organism can convert food to body mass, and is also known in the cattle industry as feed conversion efficiency. Transfer factor compositions and formulations have been successfully used to increase the body weight of cattle at an enhanced rate as compared to non-treated cattle, even in situations where the treated cattle are diseased. Accordingly, the compositions and formulations are not limited to prophylaxis and treatment of pathologies, but find use in other aspects of overall organismal health and development. In certain embodiments, methods of improving feed conversion in a subject comprise the administration to the subject of compositions and formulations comprising lyophilized transfer factor. In certain embodiments, methods of improving feed conversion in a subject comprise the administration of compositions and formulations to a subject comprising transfer factor in combination with an antibody or antibody fraction.

The transfer factor formulations of the present invention include pharmaceutical compositions suitable for administration. In a preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as sodium acetate; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the pharmaceutical compositions may be added in a micellular formulation; see U.S. Pat. No. 5,833,948, hereby expressly incorporated by reference in its entirety.

In one aspect, the components of the compositions and pharmaceutical formulations of the present invention may have an effect upon administration individually, such as for example the reduction of a tumor, but upon administration in one or more combinations, have an effect that is synergistic. By “synergistic” is meant an enhancement of the effect of one or more combined components in a more than additive fashion relative to the effect of each component when used alone.

### Method of Detecting Assaying Activity of Active Agents

According to certain aspects of the invention, quality control methods are employed to assess whether the compositions and/or formulations of the present invention stimulate lymphocyte activation. Lymphocyte function in response to antigens or mitogens may be measured by several techniques known to those skilled in the art. For example, it is known in the art that upon an appropriate stimuli, certain T lymphocytes are activated and expand their population. The expansion of this subset of lymphocytes reactive to the particular stimuli are characterized by various cellular events in the expanding cells. The events include without limitation, increased synthesis of ATP, NADP, and Proliferating Cell Nuclear Antigen (PCNA). Such intracellular components may be used to correlate the activation of the T lymphocytes as described, for example, in U.S. Pat. No. 6,630,316 (the '316 patent) to Wier, which is incorporated herein by reference. In certain embodiments of the present invention, compositions and/or formulations as described herein may be assayed for lymphocyte activation by methods, such as those described in the '316 patent.

In certain embodiments, the present invention provides methods involving administration of compositions and/or formulations according to the invention to a subject. As used herein, the term “subject” is used to mean an animal, including, without limitation, an avian or a mammal. Mammalian subjects include, without limitation, primates, bovines, porcines, ovines, equines, and carnivores, including, but not limited to, felines and canines. The mammal may be a human.

Combinations of pharmaceutical compositions may be administered. Moreover, the compositions may be administered in combination with other therapeutics.

A daily dosage of 141 mg per pound of body weight of any of the formulations in column 5 of Tables 2, 3 or 4, for 14 days has been successful in treating feline pneumonitis, feline leukemia, feline autoimmune dysfunction, feline flea bit dermatitis, feline hyperthyroidism, feline viral infection, feline ulcerations, feline bacterial infection, canine flea bite dermatitis, canine Cushings disease, malignant tumors, canine autoimmune dysfunction, canine viral and bacterial infection. These treatments for the most part have resulted in complete cures. The use of lyophilized transfer factor in these formulations is expected to produce the same or better results. In other embodiments, transfer factor in combination with antibody or antibody fraction may be used in these formulations and is expected to produce the same or better results.

Administering a formulation comprising all of the nutraceuticals in Table 2 at the preferred dosage to an animal with benign tumors resulted in about a 60% reduction in the size of the benign tumors and about a 90% reduction in the symptoms exhibited by the animal suffering the above listed diseases and syndromes. The use of lyphilized transfer factor in these formulation is expected to produce the same or better results. In other embodiments, transfer factor in combination with antibody or antibody fraction may be used in these formulations and is expected to produce the same or better results.

Administration of all of the nutraceuticals in Table 2 at the low dosage in column 3 of those tables results in about a 7% to 100% reduction in the size of the tumors and/or a 30% to 100% reduction in the symptoms exhibited by the animal suffering from those diseases or syndromes. The use of lyophilized transfer factor in these formulations is expected to produce the same or better results. In other embodiments, transfer factor in combination with antibody or antibody fraction may be used in these formulations and is expected to produce the same or better results.

The stress formulation in Table 5 is also used to treat numerous animal diseases and syndromes and as stated previously, mainly their acute stages. This formulation is also water soluble so it can be given in the animal's drinking water. A mixture of about 0.75 mg/lb transfer factor and about 1.42 mg/lb lactobacillus acidophilus 109 colony forming units (CFU) given twice daily will result in at least a 30% reduction in clinical symptoms resulting from strangles, dust cough, hypothyroidism and lymphopenia. The same dosage given to young calves will also reduce morbidity by about 30%. The addition of ionic salts or chelates of calcium, magnesium sodium and potassium twice daily in amounts approximating those in column 4 of Table 5 to the above amounts of transfer factor and lactic acid generating bacterial results in a 40% reduction in clinical symptoms of the above mentioned diseases. The addition of about 0.482 mg/lb of citric acid to the above formulation results in about a 45% reduction in the symptoms of the above mentioned diseases. Further addition of Vitamins A, B2, B6, B 12, C and E, and thiamine results in a 50% reduction in the symptoms of these diseases. The stress formulations given once or twice a day in the dosage presented in column 4 of Table 5 will cure or at least treat and reduce the symptoms of autoimmune dust cough, diarrhea from viral etiology, abscessation, in strangles, snotty nose in strangles, acute viremia in swine, scratches in the horse, hypersensitivity from scratches and onchoceriasis, PURRS, BRD, calf dysentery, coliform infections, Rhodococcus infections, Clostidium infections, circo virus in birds, and pnemonitis in cats. A combination of transfer factor and lactic acid producing bacteria or this combination further combined with yeast as shown in Table 5 will also treat these diseases but to a lesser extent. The use of lyophilized transfer factor is expected to produce the same or better results. In other embodiments, transfer factor in combination with antibody or antibody fraction may be used in these formulations and is expected to produce the same or better results.

The stress formulation as shown in Table 5 given once or twice daily will also increase the weight gain and feed efficiency of livestock. The weight gain will increase by at least 8%. A combination of transfer factor and lactic acid producing bacteria or this combination further combined with yeast as shown in Table 5 will also increase weight gain but to a lesser extent. The use of lyophilized transfer factor is expected to produce the same or better results. In other embodiments, transfer factor in combination with antibody or antibody fraction may be used in these formulations and is expected to produce the same or better results.

In a preferred embodiment, 2 gm of encapsulated hybrid glucan containing 1 gm of hybrid glucan is used.

Table 6 shows a breakdown of a performance formulation of transfer factor and nutraceuticals for treating and curing numerous diseases such as arthritis, laminitis, inflammation and malignant tumors. These diseases may also be treated with a combination of transfer factor and super oxide dismutase; transfer factor and glucosamine salts; transfer factor, glucosamine salts and super oxide dismutase; transfer factor, glucosamine salts, super oxide dismutase and glycine; transfer factor, glucosamine salts, super oxide dismutase, glycine and methyl sulfonyl methane; transfer factor, glucosamine salts, super oxide dismutase, glycine, methyl sulfonyl methane and octocosonol or transfer factor, glucosamine salts, super oxide dismutase, glycine, methyl sulfonyl methane, octocosonol and montmorillinite.

Table 7 shows a formula containing transfer factor and glucan both hybridized and non-hybridized.

Any of the aforementioned formulations may include lyophilized components, such as, for example, lyophilized transfer factor and/or lyophilized antibody or antibody fraction. Any of the aforementioned formulations may include an antibody or antibody fraction along with transfer factor.

TABLE 1
Montmorillonite Components
Average Nutrient Content Per Ounce
(1 Tablespoon = ~0.36 oz.)
(mg)

| | | | |
|---|---|---|---|
| Silicon | 6933 | Tungsten | 0.218 |
| Aluminum Silica | 2505 | Vanadium | 0.215 |
| Sodium Chloride | 1320 | Ruthenium | 0.210 |
| Potassium | 1293 | Baron | 0.189 |
| Protein | 1116 | Bromine | 0.140 |
| Calcium | 1104 | Cobalt | 0.129 |
| Sulfur | 431 | Selenium | 0.110 |
| Iron | 431 | Syprosium | 0.107 |
| Magnesium | 224 | Fluorine | 0.102 |
| Chlorine | 164 | Scandium | 0.0997 |
| Titanium | 61.9 | Samarium | 0.0943 |
| Carbon | 48.2 | Nobelium | 0.0754 |
| Sodium | 37.2 | Copper | 0.0593 |
| Barium | 10.5 | Praseodymium | 0.0539 |
| Phosphate | 8.62 | Erbium | 0.0539 |
| Strontium | 6.46 | Hafnium | 0.0539 |
| Cesium | 4.93 | Ytterbium | 0.0377 |
| Manganese | 4.04 | Lithium | 0.0377 |
| Thorium | 2.69 | Yttrium | 0.0323 |
| Uranium | 2.69 | Holmium | 0.0296 |
| Arsenic | 1.97 | Cadmium | 0.0296 |
| Chromium | 1.89 | Palladium | 0.0189 |
| Molybdenum | 1.64 | Terbium | 0.0161 |
| Nickel | 1.62 | Thulium | 0.0161 |
| Iodine | 1.28 | Gold | 0.0161 |
| Lead | 1.17 | Tantalum | 0.0135 |
| Cerium | 1.08 | Iridium | 0.0135 |
| Rubidium | 0.983 | Lutetium | 0.0108 |
| Antimony | 0.781 | Europium | 0.0108 |
| Gallium | 0.673 | Rhodium | 0.0108 |
| Germanium | 0.673 | Tin | 0.0108 |
| Neodymium | 0.539 | Silver | 0.00808 |
| Zinc | 0.539 | Indium | 0.00808 |
| Lanthanum | 0.486 | Oxygen | 0.00539 |
| Bismuth | 0.385 | Mercury | 0.00269 |
| Zirconium | 0.269 | Tellurium | 0.00269 |
| Rhenium | 0.269 | Beryllium | 0.00269 |
| Thallium | 0.269 | | |

TABLE 2
Premix Formulation
(Amounts in mg/lb of body weight unless otherwise stated)

| | | | | | | | Dosage: | |
|---|---|---|---|---|---|---|---|---|
| | | | | | | | mg/5 oz. | |
| Component | High | | Low | | Preferred | | of formula | |
| l-Arginine | 0.5 | | 0.005 | | 0.05 | | 50.00 | |
| *Lacto yeast (4.9% of blend) | 69.51 | | 0.6951 | | 6.91 | | 6951.88 | |
| Montmorillinite | 1 | gm/lb | 0.24118 | | 2.4118 | | 2411.88 | |
| Canola oil (14.5% mix) | 1.5 | gm/lb | 2.05 | | 20.571 | | 20571.88 | |
| Safflower oil (14.5% mix) | 1.5 | gm/lb | 2.05 | | 20.57 | | 20571.88 | |
| Flax seed oil (55% Alpha Linolenic | 1.5 | gm/lb | 2.05 | | 20.571 | | 1418.75 | |
| Acid) (1.0% mix) | | | | | | | | |
| Phosphorous (Monosodium | 15.750 | gm | 0.0525 | | 5.08 | | 5080.00 | |
| phosphate) 12% | | | | | | | | |
| Calcium carbonate 8.5% | 13.68 | gm | 0.0485 | | 4.88 | | 4880.00 | |
| (38% calcium) | | | | | | | | |
| Methyl sulfonyl methane | 20 | | 0.02 | | 2 | | 2000.00 | |
| Transfer factor | 50.00 | | 0.05 | | 0.75 | | 750.00 | |
| Vitamin C (ascorbic acid) | 21.62 | | 0.2162 | | 2.162 | | 2162.50 | |
| d-Biotin (Vitamin H 2%) | 9.73 | | 0.000973 | | 0.00973 | | 10.00 | |
| Vitamin D₃ | 29.16 | IU | 0.7298 | IU | 7.298 | IU | 7298.38 | IU |
| Vitamin B₁₂ | 0.092 | | 0.000092 | | 0.00092 | | 0.92 | |
| Folic Acid | 1 | | 0.001006 | | 0.01006 | | 10.06 | |
| Niacinimide | 12 | | 0.012157 | | 0.12157 | | 121.57 | |
| Pantothenic acid (d-Calcium | 0.324 | | 0.0108 | | 0.108 | | 108.00 | |
| Pantothenate) 91.6% | | | | | | | | |
| Vitamin B₆ (Pyridine Hcl) 82.3%) | 1.158 | | 0.001158 | | 0.01158 | | 11.58 | |
| Vitamin A (Retinol Palmitate) 650M | 600 | IU | 4.02 | IU | 40.212 | IU | 40232.50 | IU |
| IU/g feed grade | | | | | | | | |
| Vitamin B₂ | 0.0554 | | 0.002776 | | 0.02776 | | 27.76 | |
| Thiamine (Mononitrate) 83% | 3.09 | | 0.00308 | | 0.0308 | | 30.80 | |
| Vitamin E | 72.9 | IU | 0.0729 | IU | 0.729 | IU | 729.42 | IU |
| Vitamin K | 1 | | 0.0007 | | 0.007 | | 7.00 | |
| Cobalt (Proteinate) 5% | 0.00043 | | 0.000043 | | 0.00043 | | 0.43 | |
| Copper (Proteinate) 10% | 0.56 | | 0.0112 | | 0.112 | | 112.00 | |
| Iodine (Potassiumiodide) 98% | 0.005 | | 0.000053 | | 0.00053 | | 0.53 | |
| Iron (Proteinate) 15% | 3.31 | | 0.0331 | | 0.331 | | 331.16 | |
| Magnesium (Oxide) 58% | 10 | | 0.04 | | 0.4 | | 400.00 | |
| Manganese (Proeinate) 15% | 1.65 | | 0.04 | | 0.4 | | 332.10 | |
| Molybdenum (Sodium Molybdate | 0.05 | | 0.001 | | 0.01 | | 10.00 | |
| Dihydrate) 39% | | | | | | | | |
| Selenium (Sodium Selenite) 44.8% | 0.00162 | | 0.000081 | | 0.00081 | | 1.00 | |
| Zinc (Proteinate) 15% | 50 | | 0.04987 | | 0.4987 | | 498.72 | |
| l-Lysine (Mono HCl) | 8.41 | | 0.0841 | | 0.841 | | 841.57 | |
| d,l-Methionine | 11.03 | | 0.1103 | | 1.103 | | 1103.86 | |
| Mixed Tocopherols | | | | | | | 300.00 | |
| Choline Chloride | | | | | | | 2434.00 | |
| Sipernat 50 (Silicon dioxide) | | | | | | | 12768.75 | |
| Lodex-5 (maltodextrin) | | | | | | | 7519.38 | |
| Soy flour (17.5% mix) | | | | | | | 24828.13 | |
| Sweet whey | | | | | | | 996.00 | |
| BF70 spice | | | | | | | 146.00 | |
| Dextrose powder | | | | | | | 750.00 | |

*Lactic acid generating bacteria is two-thirds of component and yeast is one-third; lactic acid generating bacteria is 500,000,000 CFU/gm, yeast (e.g., “Saccharamyces”) 250,000,000 CFU/gm

TABLE 3
Canine Premix Formulation
(Amounts in mg/lb of body weight unless otherwise stated)

| | | | | |
|---|---|---|---|---|
| | | | | Dosage: mg/oz |
| Component | High | Low | Preferred | of formula |

| | | | | | | | | |
|---|---|---|---|---|---|---|---|---|
| l-Arginine | 0.5 | | 0.005 | | 0.05 | | 10.00 | |
| *Lacto yeast (4.9% of blend) | 69.51 | | 0.6951 | | 6.91 | | 1390.38 | |
| Montmorillinite | 1 | gm/lb | 0.24118 | | 2.4118 | | 482.20 | |
| Canola oil (14.5% mix) | 1.5 | gm/lb | 2.05 | | 20.571 | | 3887.00 | |
| Safflower oil (14.5% mix) | 1.5 | gm/lb | 2.05 | | 20.57 | | 3887.00 | |
| Flax seed oil (55% Alpha | 1.5 | gm/lb | 2.05 | | 20.571 | | 240.00 | |
| Linolenic Acid) (1.0% mix) | | | | | | | | |
| Phosphorous (Monosodium | 15.750 | gm | 0.0525 | | 5.08 | | 1010.00 | |
| phosphate) 12% | | | | | | | | |
| Calcium carbonate 8.5% | 13.68 | gm | 0.0485 | | 4.88 | | 977.00 | |
| (38% calcium) | | | | | | | | |
| Methyl sulfonyl methane | 20 | | 0.02 | | 2 | | 400.00 | |
| Transfer factor | 50.00 | | 0.05 | | 2.50 | | 500.00 | |
| Vitamin C (ascorbic acid) | 21.62 | | 0.2162 | | 2.162 | | 432.50 | |
| d-Biotin (Vitamin H 2%) | 9.73 | | 0.000973 | | 0.00973 | | 2.00 | |
| Vitamin D₃ | 29.16 | IU | 0.7298 | IU | 7.298 | IU | 1459.68 | IU |
| Vitamin B₁₂ | 0.092 | | 0.000092 | | 0.00092 | | 0.18 | |
| Folic Acid | 1 | | 0.001006 | | 0.01006 | | 2.16 | |
| Niacinimide | 12 | | 0.012157 | | 0.12157 | | 24.31 | |
| Pantothenic acid (d-Calcium | 0.324 | | 0.0108 | | 0.108 | | 21.60 | |
| Pantothenate) 91.6% | | | | | | | | |
| Vitamin B₆ (Pyridine Hcl) 82.3%) | 1.158 | | 0.001158 | | 0.01158 | | 2.32 | |
| Vitamin A (Retinol Palmitate) | 600 | IU | 4.02 | IU | 40.212 | IU | 8046.50 | IU |
| 650 M IU/g feed grade | | | | | | | | |
| Vitamin B₂ | 0.0554 | | 0.002776 | | 0.02776 | | 5.55 | |
| Thiamine (Mononitrate) 83% | 3.09 | | 0.00308 | | 0.0308 | | 0.16 | |
| Vitamin E | 72.9 | IU | 0.0729 | IU | 0.729 | IU | 145.88 | IU |
| Vitamin K | 1 | | 0.0007 | | 0.007 | | 1.40 | |
| Cobalt (Proteinate) 5% | 0.00043 | | 0.000043 | | 0.00043 | | 0.086 | |
| Copper (Proteinate) 10% | 0.56 | | 0.0112 | | 0.112 | | 22.40 | |
| Iodine (Potassiumiodide) 98% | 0.005 | | 0.000053 | | 0.00053 | | 0.106 | |
| Iron (Proteinate) 15% | 3.31 | | 0.0331 | | 0.331 | | 66.23 | |
| Magnesium (Oxide) 58% | 10 | | 0.04 | | 0.4 | | 80.00 | |
| Manganese (Proeinate) 15% | 1.65 | | 0.04 | | 0.4 | | 66.42 | |
| Molybdenum (Sodium Molybdate | 0.05 | | 0.001 | | 0.01 | | 2.00 | |
| Dihydrate) 39% | | | | | | | | |
| Selenium (Sodium Selenite) | 0.00162 | | 0.000081 | | 0.00081 | | 0.20 | |
| 44.8% | | | | | | | | |
| Zinc (Proteinate) 15% | 50 | | 0.04987 | | 0.4987 | | 99.74 | |
| l-Lysine (Mono HCl) | 8.41 | | 0.0841 | | 0.841 | | 176.91 | |
| d,l-Methionine | 11.03 | | 0.1103 | | 1.103 | | 220.77 | |
| Mixed Tocopherols | | | | | | | 60.00 | |
| Choline Chloride | | | | | | | 486.80 | |
| Sipernat 50 (Silicon dioxide) | | | | | | | 2553.35 | |
| Lodex-5 (maltodextrin) | | | | | | | 1508.87 | |
| Peanut oil | | | | | | | 496.56 | |
| Soy flour (17.5% mix) | | | | | | | 4965.02 | |
| Peanut flour | | | | | | | 4965.02 | |
| Sweet whey | | | | | | | 400.00 | |
| BF70 spice | | | | | | | 29.20 | |
| Dextrose powder | | | | | | | 500.00 | |

*Lactic acid generating bacteria is two-thirds of component and yeast is one-third; lactic acid generating bacteria is 500,000,000 CFU/gm, yeast (e.g., “Saccharamyces”) 250,000,000 CFU/gm

TABLE 4
Feline Premix Formulation
(Amounts in mg/lb of body weight unless otherwise stated)

| | | | | | | | Dosage: | |
|---|---|---|---|---|---|---|---|---|
| | | | | | | | mg/2.2 gm | |
| Component | High | | Low | | Preferred | | of formula | |
| l-Arginine | 0.5 | | 0.005 | | 0.05 | | 0.78 | |
| *Lacto yeast (4.9% of blend) | 69.51 | | 0.6951 | | 6.91 | | 108.42 | |
| Montmorillinite | 1 | gm/lb | 0.24118 | | 2.4118 | | 37.00 | |
| Canola oil (14.5% mix) | 1.5 | gm/lb | 2.05 | | 20.571 | | 323.25 | |
| Safflower oil (14.5% mix) | 1.5 | gm/lb | 2.05 | | 20.57 | | 323.25 | |
| Flax seed oil (55% Alpha | 1.5 | gm/lb | 2.05 | | 20.571 | | 22.13 | |
| Linolenic Acid) (1.0% mix) | | | | | | | | |
| Phosphorous (Monosodium | 15.750 | gm | 0.0525 | | 5.08 | | 78.70 | |
| phosphate) 12% | | | | | | | | |
| Calcium carbonate 8.5% | 13.68 | gm | 0.0485 | | 4.88 | | 75.69 | |
| (38% calcium) | | | | | | | | |
| Methyl sulfonyl methane | 20 | | 0.02 | | 2 | | 31.20 | |
| Transfer factor | 50.00 | | 0.05 | | 16.00 | | 250.00 | |
| Vitamin C (ascorbic acid) | 21.62 | | 0.2162 | | 2.162 | | 33.73 | |
| d-Biotin (Vitamin H 2%) | 9.73 | | 0.000973 | | 0.00973 | | 0.156 | |
| Vitamin D₃ | 29.16 | IU | 0.7298 | IU | 7.298 | IU | 113.90 | IU |
| Vitamin B₁₂ | 0.092 | | 0.000092 | | 0.00092 | | 0.014 | |
| Folic Acid | 1 | | 0.001006 | | 0.01006 | | 0.168 | |
| Niacinimide | 12 | | 0.012157 | | 0.12157 | | 1.90 | |
| Pantothenic acid (d-Calcium | 0.324 | | 0.0108 | | 0.108 | | 1.68 | |
| Pantothenate) 91.6% | | | | | | | | |
| Vitamin B₆ (Pyridine Hcl) 82.3%) | 1.158 | | 0.001158 | | 0.01158 | | 0.18 | |
| Vitamin A (Retinol Palmitate) | 600 | IU | 4.02 | IU | 40.212 | IU | 627.60 | IU |
| 650 M IU/g feed grade | | | | | | | | |
| Vitamin B₂ | 0.0554 | | 0.002776 | | 0.02776 | | 0.43 | |
| Thiamine (Mononitrate) 83% | 3.09 | | 0.00308 | | 0.0308 | | 0.48 | |
| Vitamin E | 72.9 | IU | 0.0729 | IU | 0.729 | IU | 11.38 | IU |
| Vitamin K | 1 | | 0.0007 | | 0.007 | | 0.11 | |
| Cobalt (Proteinate) 5% | 0.00043 | | 0.000043 | | 0.00043 | | 0.006 | |
| Copper (Proteinate) 10% | 0.56 | | 0.0112 | | 0.112 | | 1.75 | |
| Iodine (Potassiumiodide) 98% | 0.005 | | 0.000053 | | 0.00053 | | 0.008 | |
| Iron (Proteinate) 15% | 3.31 | | 0.0331 | | 0.331 | | 5.17 | |
| Magnesium (Oxide) 58% | 10 | | 0.04 | | 0.4 | | 6.24 | |
| Manganese (Proeinate) 15% | 1.65 | | 0.04 | | 0.4 | | 5.18 | |
| Molybdenum (Sodium Molybdate | 0.05 | | 0.001 | | 0.01 | | 0.156 | |
| Dihydrate) 39% | | | | | | | | |
| Selenium (Sodium Selenite) | 0.00162 | | 0.000081 | | 0.00081 | | 0.156 | |
| 44.8% | | | | | | | | |
| Zinc (Proteinate) 15% | 50 | | 0.04987 | | 0.4987 | | 7.78 | |
| l-Lysine (Mono HCl) | 8.41 | | 0.0841 | | 0.841 | | 13.80 | |
| d,l-Methionine | 11.03 | | 0.1103 | | 1.103 | | 17.22 | |
| Mixed Tocopherols | | | | | | | 4.68 | |
| Choline Chloride | | | | | | | 38.0 | |
| Sipernat 50 (Silicon dioxide) | | | | | | | 199.06 | |
| Lodex-5 (maltodextrin) | | | | | | | 117.30 | |
| Sweet whey | | | | | | | 155.37 | |
| BF70 spice | | | | | | | 2.28 | |
| Dextrose powder | | | | | | | 250.00 | |
| Glucosamine HCl | | | | | | | 100.00 | |
| Pernaconniculus-Chondroitin | | | | | | | 200.00 | |

*Lactic acid generating bacteria is two-thirds of component and yeast is one-third; lactic acid generating bacteria is 500,000,000 CFU/gm, yeast (e.g., “Saccharamyces”) 250,000,000 CFU/gm

TABLE 5
Stress Formula
(Amounts in mg/lb of body weight unless otherwise stated)

| | | | | | | Dosage: |
|---|---|---|---|---|---|---|
| | | | | | | mg/ounce |
| Component | High | | Low | | Preferred | of formula |
| Calcium | 1.80 | | 0.09 | | 0.028 | 28.00 |
| Pantothenate | | | | | | |
| Vitamin C | 20.00 | | 0.056 | | 0.017 | 17.00 |
| (ascorbic acid) | | | | | | |
| Vitamin B₁₂ | 13.00 | | 0.13 | | 0.198 | 198.59 |
| Vitamin A | 600.00 | IU | 0.10 | IU | 0.014 | 14.00 |
| Vitamin B₂ | 1.20 | | 0.065 | | 0.018 | 18.00 |
| Thiamine | 16.00 | | 0.0308 | | 0.017 | 17.00 |
| Vitamin E | 72.9 | IU | 0.729 | IU | 0.012 | 12.48 |
| Magnesium Sulfate | 10.00 | | 0.113 | | 0.113 | 113.00 |
| *Lactobacillus | 10.00 | | 0.467 | | 1.418 | 1418.00 |
| acidophilus | | | | | | |
| Sodium | 166.00 | | 0.236 | | 2.368 | 2368.00 |
| Chloride | | | | | | |
| Dipotassium | 116.00 | | 5.85 | | 1.773 | 1773.00 |
| phosphate | | | | | | |
| Citric acid | 31.00 | | 1.59 | | 0.482 | 482.00 |
| Yeast | 180.00 | | 0.1957 | | 0.283 | 283.00 |
| (hydrolyzed) | | | | | | |
| Glycine | 0.142 | | 0.0142 | | 0.142 | 141.80 |
| Potassium | 18.00 | | 0.93 | | 0.283 | 283.00 |
| chloride | | | | | | |
| Vitamin D₃ | 29.00 | | 0.729 | | 0.002 | 1.56 |
| Dextrose | 40.00 | | 2.00 | | 21.38 | 21375.00 |
| Artificial flavor | 0.028 | | 0.0028 | | 28.548 | 28.30 |
| Transfer Factor | 50.00 | | 0.05 | | 0.75 | 750.00 |
| Sipernat | | | | | 0.05 | 56.70 |
| (silicon dioxide) | | | | | | |

*10⁹ colony forming units (CFU)/gm

TABLE 6
Performance Formula
(Amounts in mg/lb of body weight unless otherwise stated)

| | | | | Dosage: |
|---|---|---|---|---|
| | | | | mg/oz. |
| Component | High* | Low* | Average* | of formula |
| Super oxide dismutase | 60.0 | 0.6 | 6.0 | 6000.0 |
| Glucosamine salts | 65.0 | 0.65 | 6.5 | 6500.0 |
| Transfer factor¹ (horses, cows) | 15.0 | 0.15 | 1.5 | 1500.0 |
| Transfer factor¹ (goats) | 10.0 | 0.10 | 1.0 | 3000.0 |
| Transfer factor¹ (dogs, cats) | 50.0 | 0.5 | 5.0 | 14000.0 |
| Pernaconniculus-Chondroitin | 16.5 | 0.165 | 1.65 | 1650.0 |
| (mucopolysaccharides) | | | | |
| Boswellic acids | 30 | 0.3 | 3.0 | 3000.0 |
| Di-methyl glycine | 27.0 | 0.27 | 2.7 | 2700.0 |
| Methyl sulfonyl methane | 27.0 | 0.27 | 2.7 | 2700.0 |
| Octocosonol | 2.0 | 0.004 | 0.04 | 400.0 |
| Montmorillinite | 30.0 | 0.3 | 3.0 | 3000.0 |

*These amounts are calculated for livestock animals weighing about 450 to 1,000 pounds, goats weighing about 150 pounds, and dogs and cats weighing from about 8 to about 15 pounds.
¹The amount of transfer factor may vary for different species but the amounts for the other components remain the same for each species.

TABLE 7
Livestock Stress Rumen By-Pass
(Amounts in mg/lb of body weight unless otherwise stated)

| | Dosage: mg/oz. | |
|---|---|---|
| | (unless otherwise | |
| Component | noted) of formula | |
| Stabilized¹ | | |
| Transfer factor (mammal source) | 3500.0 | |
| Transfer factor (avian source) | 1000.0 | |
| β-sitosterol (90% phytosterols) | 300.0 | |
| Inositol hexaphosphate | 350.0 | |
| Olive leaf extracts | 35.0 | |
| Aloe extract powder (200:1) | 17.0 | |
| Hybridized and non-hybridized | 4000.0 | |
| Glucans (from Hybridized Cordycepts | | |
| sinensis, Agaricus blazeii, Miatake, | | |
| Shitake, Coriolis, Inonotus, Obliquus, and | | |
| Poris cocos mushrooms) | | |
| Vitamin C | 2000.0 | |
| Non-Stabilized | | |
| Vitamin A | 4434 | IU/oz |
| Vitamin D3 | 1140 | IU/oz |
| Vitamin E | 500 | IU/oz |
| Vitamin B1 | 12.77 | |
| Vitamin B2 | 12.77 | |
| Vitamin B12 | 1.5 | |
| Di-potassium phosphate | 1.5 | g/oz |
| Potassium chloride | 207 | |
| Magnesium sulfate | 83 | |
| Calcium pantothenate | 23 | |
| Ascorbic acid | 23 | |
| Lactic acid bacteria | 2.5 × 10⁶ | CFU/oz |
| Yeast (S. cerivisiea) | 15.0 × 10⁶ | CFU/oz |
| Zinc proteinate | 10 | |

*These amounts are calculated for livestock animals weighing about 450 to 1,000 pounds, goats weighing about 150 pounds, and dogs and cats weighing from about 8 to about 15 pounds.
¹Stabilized active ingredients are included in a formulation of 50% soybean oil and 50% active ingredient.

### Kits

In one aspect, the present invention provides kits suitable for treatment of an subject, for example, an animal. The kits may further include instructions for use. Instructions may be included as a separate insert and/or as part of the packaging or container, such as a label affixed to a container or as a writing or other communication integrated as part of a container. The instructions may inform the user of methods of administration of the compositions and formulations contained therein, precautions, expected results, warnings concerning improper use, and the like.

In one embodiment, the kit includes a first container having a composition or a formulation that includes a transfer factor. In addition to the transfer factor, the formulations and compositions of the present invention may also include an antibody or antibody fraction. In certain embodiments, the antibody or antibody fraction may be present at about 5% to about 35% of the formulation or composition. In other embodiments, the antibody or antibody fraction is present at about 10% to about 30%, from about 15% to about 25%, from about 17.5% to about 22.5%, or about 20%. The first container of the present invention may contain compositions or formulations that have either (1) transfer factor or (2) transfer factor and antibody or antibody fraction in a lyophilized form.

In another aspect, the kits provide a first container having a composition or formulation of the present invention that is encapsulated by a hydrophobic or lipid coating. In another embodiment, the hydrophobic or lipid coating may include essential fats and/or plant oils. The plant oil may be soybean oil.

In another aspect, the kit may include a composition or formulation containing a glucan, including without limitation a hybrid glucan, a hydrolyzed glucan, and a hydrolyzed hybrid glucan, as described herein. The glucan may be derived from a fungus. The fungus may be a whole fungus. As described herein, the glucan may be derived from a Cordyceps strain, including without limitation the Cordyceps sinensis strain. It may also be a hybrid glucan, as described herein. In one embodiment, the glucan is encapsulated by a hydrophobic or lipid coating as described herein. The coating may include an essential fat and/or a plant oil. The plant oil may be soybean oil. The glucan may be hydrolyzed as described herein. In some embodiments, the glucan is provided in the first container, which already contains the transfer factor. However, the glucan may also be provided in a second container separate from the first container. The composition or formulation having the glucan may include a lyophilized glucan.

The present invention provides kits having a first container with a transfer factor and a second container with a glucan. The transfer factor may be lyophilized. If the transfer factor and/or the glucan is lyophilized, the kit may further comprise a third container having a reconstitution solution for reconstitution of the lyophilisate(s). The reconstitution solution may be any solution suitable for reconstituting a lyophilistate known to those skilled in the art. For example, water or any suitable solvent may be used. In addition, the kit may contain instructions for the reconstitution of the lyophilisates in the reconstitution solution.

In another aspect, the kit includes instructions for the administration of the formulations and/or compositions included in the containers described herein. The kits as described herein may further comprise suitable packaging of the respective compositions, instructions, and/or other optional components. In one embodiment, kits of the present invention may further contain components useful in the application of the compositions and formulations described herein.

In one aspect, the kit includes instructions for the prevention and/or treatment of a condition in a subject. The conditions suitable for treatment are described herein, including without limitation, benign and malignant tumors. The present invention includes methods, compositions, and pharmaceutical formulations for the prevention and/or treatment of conditions and/or diseases in a subject, including a mammal. Included are methods, compositions and formulations suitable for animals, including mammals, including humans.

### Compositions, Pharmaceutical Formulations and Methods of Treatment

In certain embodiments, the present invention provides methods of treating a subject with a condition by administering a composition or a pharmaceutical formulation, as described herein. In one embodiment, the pharmaceutical formulation is prepared from a kit described herein. The compositions may contain one or more of the following components, in any combination: transfer factor, lyophilized transfer factor, an antibody or antibody fraction, a lyophilized antibody or antibody fraction, and a glucan. For example, the composition or pharmaceutical formulation may comprise (i) a transfer factor, (ii) a transfer factor and an antibody or antibody fraction (iii) a transfer factor, an antibody or antibody fraction and a glucan, (iv) a lyophilized transfer factor and an antibody or antibody fraction (v) a lyophilized transfer factor and a lyophilized antibody or antibody fraction, (vi) a lyophilized transfer factor, a lyophilized antibody or antibody fraction, and a glucan. In certain embodiments, the compositions and formulations prepared from a kit may optionally include additional components. In one embodiment, the transfer factor of any of these combinations may be lyophilized. In other embodiments, the antibody or antibody fraction of a composition or pharmaceutical formulation described herein may be lyophilized.

In another aspect, the compositions and pharmaceutical formulations provided by the present invention comprise glucans from a particular strains of fungus. In one embodiment, a glucan is derived from one or more strains of fungus. By “fungus” herein is meant a fungus other than yeast unless otherwise noted. The present invention contemplates the derivation of one or more glucans, hybrid glucans, hydrolyzed glucans, and hydrolyzed hybrid glucans from one of the following strains: Cordyceps sinensis, Agaricus blazeii, Miatake, Shiake, Coriolis, Inonotus, Obliquus, and Poris cocos. In another embodiment, glucans are derived from more than one strain. U.S. Patent Application Publication No. 2006/0073197 A1, which is incorporated herein by reference in its entirety, also relates to the glucan-containing compositions and pharmaceutical formulations of the present invention.

In one embodiment, the compositions and pharmaceutical formulations of the present invention include one or more of the following: lyophilized transfer factor, a hybrid glucan, a hydrolyzed hybrid glucan, and an antibody or antibody fraction. Glucans may be derived from different sources, including various species of fungus. For example, a composition or formulation of the present invention may contain hydrolyzed glucans derived from Cordyceps sinensis, Agaricus blazei, Grifola frondosa, Ganoderma lucidum, Lentinula edodes, and/or Coriolus versicolor. In one embodiment, the glucans may be hybrid or non-hybrid glucans derived from Cordyceps sinensis.

In another embodiment, the compositions and pharmaceutical formulations containing various combinations of transfer factor and antibody or antibody fraction may further comprise one or more ingredients of the performance formula according to Table 6 above.

The conditions suitable for treatment using these compositions, pharmaceutical formulations and by these methods include, without limitation, a malignant tumor, a benign tumor, hyperthyroidism, lymphopenia, Cushing's disease, Addison's disease, weight loss, hair loss, fatigue, and anorexia.

In one embodiment, the malignant tumor may be a carcinoma, including without limitation, a squamous cell carcinoma, a transitional cell carcinoma, an adenocarcinoma or a thyroid carcinoma. Tumors may be found in any organ or tissue of the body. The primary tumor or original tumor is the place where the cancer begins but it can spread or metastasize and form metastatic tumors in other parts of the body. Carcinomas are tumors that begins in the skin or in tissues that line or cover internal organs. In some cases, the primary cancer site may not be known and the tumor is called a carcinoma of unknown primary origin. Subjects with such tumors may have a cell type called adenocarcinoma, which refers to cancer that begins in the cells in glandular structures in the lining or covering of certain organs in the body. Common primary sites for adenocarcinomas include without limitation the lung, pancreas, breast, prostate, stomach, liver, and colon.

In another embodiment, the malignant tumor may be a sarcoma, including without limitation, a fibrosarcoma, a chondrosarcoma, a lymphosarcoma, a melanosarcoma, an osteocsarcoma, or a hemangiosarcoma. In one embodiment, the melanosarcoma may be an amelanotic melanosarcoma. In one other embodiment, the hemangiosarcoma is splenic. A sarcoma includes cancers of the bone, bone, cartilage, fat, muscle, blood vessels, or other connective or supportive tissue.

In one embodiment, the malignant tumor may be a mast cell tumor. Mast cell tumors may be graded as grade 1, grade 2, grade 3, or grade 4. Grading of mast cell tumors is typically performed by a pathologist during a biopsy. The grade assigned indicates the malignant characteristic of the cells, where grade 1 is benign, grade 3 is malignant, grade 2 is between 1 and 3, and grade 4 is a rapidly growing malignant tumor with metastasis. Mast cell tumors are frequently found in canines.

In one embodiment, the malignant tumor is a histiocytoma, which may originate from a Langerhans cell found in the skin. Such cells are part of the immune system and process and present external antigens to other cells in the immune system. Histiocytomas are frequently found in canines, including without limitation Labrador retrievers, Stafforshire terriers, Boxers, and Daschunds.

In some embodiments, the malignant tumors suitable for treatment also include, without limitation, a melanoma, a bone tumor, a keratoma, a lipoma, plepharitis eye tumors, dermal tumors, and leukemia. The lymphoma may be a Hodgkin's lymphoma or a non-Hodgkin's lymphoma.

In other embodiments, the condition suitable for treatment by the methods, compositions, and pharmaceutical formulations of the present invention is a benign tumor or an adenoma, including without limitation meibomian gland adenomas, a glandular adenoma of the skin, and perinanal adenomas. In one other embodiment, the malignant tumor is a granulosa tumor of the ovary.

In another embodiment, the condition suitable for treatment by the methods, compositions, and pharmaceutical formulations of the present invention is a fungal infection, including without limitation blastomycosis and coccidiomycosis. Infection may occur by inhalation. Blastomycosis is caused by the fungus Blastomyces dermatitidis. Coccidiomycosis is caused by spores from the fungus, Coccidiodes immitis. Fungal infections may occur in subjects with compromised immune systems, including without limitation people with HIV and organ transplant recipients.

In one aspect of the present invention, the methods, compositions, and pharmaceutical formulations provide a way to reduce tumor size in a subject. In one embodiment of the invention, there is provided a method of reducing tumor size in a subject in need thereof comprising administering to the subject a composition comprising transfer factor and an antibody or antibody fraction. In another embodiment, there is provided a method of reducing tumor size in a subject in need thereof comprising administering to the subject a composition comprising lyophilized transfer factor. In certain embodiments, the tumor size may be reduced by at least about 50%, or at least about 90%.

By “reduce” or “reduction” and any grammatical equivalents, is meant a cytotoxic effect on tumor cells and/or a cytostatic effect on tumor cells, as well as the regression of a tumor, such that its size is reduced. In one embodiment, tumors are reduced from about 1% to about 100%, about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, and about 50%. In other embodiments, the tumor reduced may be a malignant or a benign tumor. Some embodiments provide a reduction of particular types of tumors. For example, a malignant tumor may be reduced from about 20% to about 40% and a benign tumor may be reduced from about 80% to about 100%. In addition, particular types of malignant tumors may be reduced. For example, an amelanotic melanosarcoma may be reduced by about 80%, a lytic bone tumor may be reduced by about 100%, a keratoma may be reduced by about 100%, a mast cell tumor may be reduced from about 50% to about 80%, an osteosarcoma may be reduced by about 80%, and a melanoma may be reduced from about 50% to about 80%. In other embodiments, the reduction may be transient. For example, a lymphoma may be transiently reduced from about 50% to about 80% and a hemangiosarcoma may be transiently reduced for about one year from about 50% to about 80%.

The following examples serve to more fully describe the manner of using the above-described invention. It is understood that these examples in no way serve to limit the true scope of this invention, but rather are presented for illustrative purposes.

### EXAMPLE 1

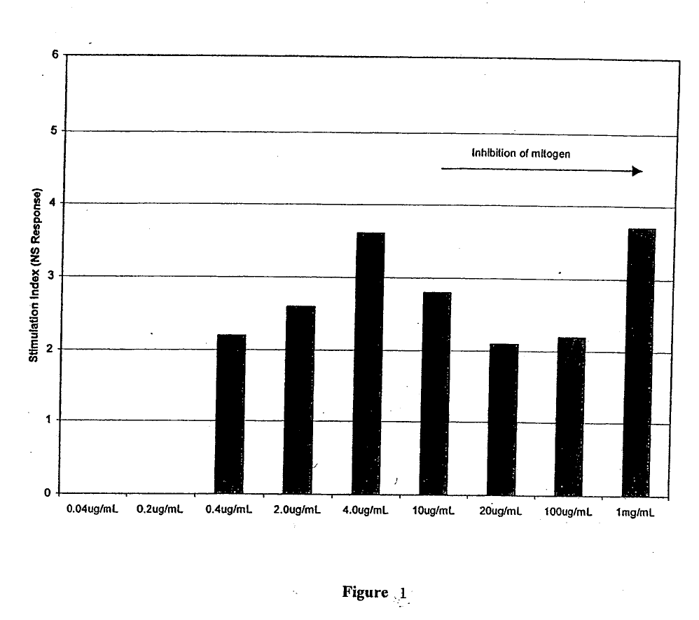

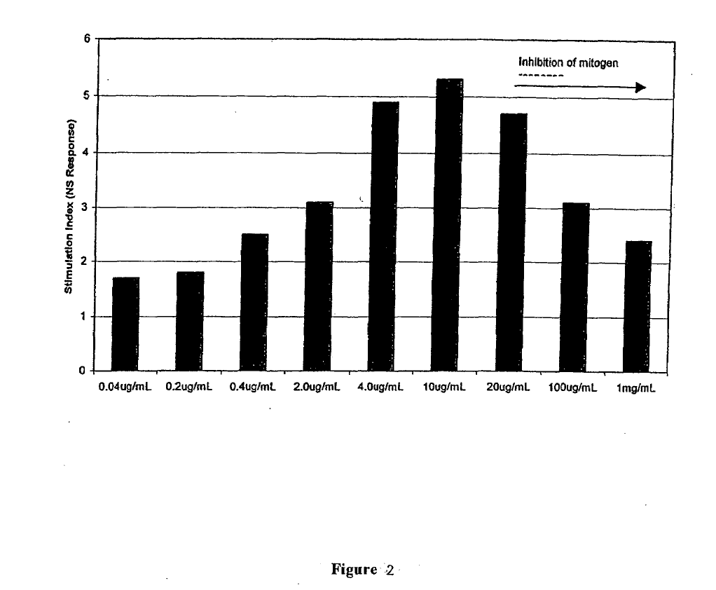

The compositions and/or formulations of the present invention may be assayed for lymphocyte stimulation activity using procedures known in the art, such as the ImmunKnow™ Immune Cell Function Assay provided by Cylex, Inc. and discussed herein (See Wier U.S. Pat. No. 6,630,316). Dilutions of the compositions and/or formulations if the present invention may be prepared as a stock solution having a concentration of 1 mg/ml and stored at 4° C. Dilutions of the stock solution can be prepared for analysis. For example, dilutions were prepared at 0.04, 0.2, 0.4, 2.0, 4.0, 10, 20, and 100 μg/mL, as well as 1 mg/mL, for a number of samples. Each diluted sample was added to a 96 well assay plate, followed by the addition of diluted whole blood to each assay well containing the diluted sample. The plates were incubated at 37° C. for one hour and then a sample diluent of Phytohemagglutinin-L (PHA) was added to each well. The plates were then incubated at 37° C. for 15-18 hours, after which magnetic beads coated with mouse monoclonal anti-human CD4 (Dynabeads®* CD4) were added. Following a 15 second agitation on a plate shaker, the plates were incubated at room temperature (18-28° C.) for 15 minutes. This step was repeated and a final agitation step on a plate shaker for 15-3-seconds was performed. The magnetic beads were then separated from the sample, followed by a washing step to remove any residual unbound cells. A lysis reagent was added to each well. A luminescent reagent was added and the amount of ATP present in each cell lysate was assessed. The results shown in FIGS. 1-2 are in terms of a stimulation index. The samples analyzed include a composition comprising a lyophilized transfer factor (FIG. 1) and a composition comprising hydrolyzed glucans and glucans (Immune-Assist as provided by Aloha Medicinals and containing Agaricus blazei, Cordyceps sinesis hybrid, Lentinula edodes, Grifola frondosa, Ganoderma lucidum, and Coriolus versicolor) (FIG. 2). Results were also obtained for a composition comprising a lyophilized transfer factor with a 20% antibody fraction, spray-dried.

### EXAMPLE 2

A calf study was performed using the stress formula disclosed in Table 5 above but containing lyophilized transfer factor. A 1-2% death loss was observed. When the same formula containing a spray-dried lyophilized transfer factor was administered, the death loss increased to 37.5%, that is, six out of sixteen animals died after being treated three times.

### EXAMPLE 3

A cow study was performed using the stress formula disclosed in Table 5 above but containing lyophilized transfer factor. An 11.7% death loss was observed. A similar study performed thereafter resulted in a 6.5% death loss. Another study was performed using the stress formula from Table 5 above but containing spray-dried transfer factor (4-Life) and a 35% death loss was observed (48 out of 137 died).

| | Head treated | dead | % | |
|---|---|---|---|---|
| January 2006 | 130 | 34 | 26% | On lyophilized transfer factors |
| February 2006 | 137 | 16 | 12% | On lyophilized transfer factors |
| March 2006 | 124 | 8 | 6% | On lyophilized transfer factor |
| April 2006 (Ran out of lyophilized | 137 | 48 | 35% | |
| transfer factors and switched | | | | |
| to spray dried product) | | | | |
| May 5, 2006 (Animals given | 20 | 1 | 5% | |
| lyophilized transfer factors) | | | | |

Their problems subsided to 3% death loss with use of lyophilized transfer factors.

### COMPARATIVE EXAMPLE

Spray dried transfer factor product was tested on 16 calves out of 100 head shipment. Sixteen calves got spray dried transfer factor on days one, two, and twelve. Results:

three died

13 head treated twice, none doing well in feed efficiency and poor hair coat

### EXAMPLE 4

baby calves two to three days old.

15 head on lyophilized transfer factor product

a) treated one with mild scours,
b) all calves sucking on own day 3
c) calves on full feed Day 7

15 head treated with spray dried transfer factor product

a) five treated calves
b) calves sucking on day 7
c) calves on full feed day 14

15 head of Controls no transfer factor.

a) treated all 15 calves over two times each
b) calves all sucking on day 10 to 14
c) calves still not on full feed day 24

### EXAMPLE 5

1500 head of dairy calves in Oakdale California was treated with spray dried transfer factor product since November, 2002, averaging about 5 to 8% death loss. When switched to lyophilized transfer factor product dropped the death loss dropped to about 2.8%.

The above Examples 2-5 demonstrate reduced mortality and morbidity in calves treated with lyophilized transfer factor in comparison with spray dried transfer factor and transfer factor-free controls.

It will be apparent to those skilled in the art that various modifications and variations can be made in the methods and compositions of the present inventions without departing from the spirit or scope of the invention. Thus, it is intended that the present invention cover the modification and variations of the inventions provided they come within the scope of the appended claims and their equivalents.

The terms and expressions which have been employed are used as terms of descriptions and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope on this invention.

In addition, where features or aspects of the invention are described in terms of Markush group or other grouping of alternatives, those skilled in the art will recognized that the invention is also thereby described in terms of any individual member or subgroup of members of the Markush group or other group.

Unless indicated to the contrary, all numerical ranges described herein include all combinations and subcombinations of ranges and specific integers encompassed therein. Such ranges are also within the scope of the described invention.

The disclosures of each patent, patent application and publication cited or described in this document are hereby incorporated herein by reference, in their entirety.

APPENDIX 1
HUMAN AND BOVINE PATHOGENS:
POTENTIONAL CROSS REACTIVITY

| Human Pathogen or Disease | Commonality | Bovine Pathogen |
|---|---|---|
| BACTERIA | | |
| Travelers Disease (E. coli) | very | Toxigenic E. coli |
| | very | Campylobacter jejuni |
| Bloody diarrhea/hemolytic uremia | increasing | E. coli 0157:H7 Verotoxic |
| Salmonellosis/Typhoid Fever | common | Salmonella thyphimurium, |
| Salmonella typhosa | | dublin |
| Diarrhea, from food or water | very | Campylobacter jejuni |
| Clostridial Infection (non-tetanus) | common | Clostridia (many species) |
| C. dificil | | |
| Mycobacterium Infections | | Mycobacterium species |
| johnei, Crohn's Disease | common | common in Jersey cattle |
| Staphylococcal super infections | common | Staph. aureus |
| Streptococcal infections | common | Streptococcus |
| Endocarditis | common | Beta Strep. |
| Superinfection | increasing | S. pyogenes |
| S. pyogenes | increasing | |
| Enterococci | common | Enterococci (most spp. & VRE) |
| Hospital/VRE strains serious | common | |
| Helicobacter pylon (ulcers) | common | Bovine/Porcine association |
| VIRUS | | |
| Influenza | common | Influenza virus |
| Pneumonia Resp. Syncytial Virus | common | Bovine Resp. Sync. Virus |
| Papilloma, Condylomaya | common | Bovine Papilloma Virus |
| Virus Diarrhea | common | Bovine Virus Diarrhea |
| Rotavirus | | Rotavirus |
| | | Coronavirus |
| Cytomegalovirus | common | Bovine CMV and IBR |
| Herpes Infections | common | Bovine Rhinotracheitis |
| HIV (Retrovirus) | common | Bovine Immune Deficiency |
| | | Virus |
| Rhinovirus (common cold) | very | Bovine Rhinovirus |
| YEAST, FUNGI and PROTOZOA | | |
| Candidiasis | common | Candida exp. common |
| Cryptosporidiosis | very | Calf diarrhea, C. parvum |
| Giardiasis | common | Calf diarrhea, G. lamblia |
| OTHER | | |
| Mycoplasma pneumonia, arthritis | common | Bvn. Mycopl. Pneumonia |

APPENDIX 2
HUMAN AND AVIAN PATHOGENS:
POTENTIAL CROSS REACTIVITY

| | Common- | |
|---|---|---|
| Human Pathogen or Disease | ality | Avian Pathogen |
| BACTERIA | | |
| Travelers Diarrhea (E. coli) | very | Toxigenic E. coli |
| | very | Campylobacter jejuni |
| Bloody diarrhea/hemolytic | increasing | E. coli 0157:H7 verotoxic |
| uremia | | |
| Diarrhea | | O1, O2, O47, others |
| Salmonellosis | very | Salmonella sp. |
| Diarrhea, from food and water | very | Campylobacter jejuni |
| Clostridial Infection | common | Clostridia sp. |
| Pasteurellosis | very | Pasteurella multocida |
| Pneumonia | common | Haemophilus gallinarium |
| | common | Mycoplasma gallispeticum |
| | common | Chlamydia pneumona |
| Systemic infection | common | Erysipeloxthrix insidiosa |
| Diarrhea, systemic infection | very | Lisreria monocytogenes |
| VIRUS | | |
| Chicken pox | very | Fowl pox |
| Influenza | very | Influenza virus |
| Infectious bronchitis | common | Infectious Bronchitis |
| Adult Leukemia virus | rare | Marek's disease virus |
| (ATLV-1) | | |
| Pneumonia | common | Paramyxovirus |
| Herpetic infections | common | Herpes simplex virus |
| FUNGAL | | |
| Pneumonia, systemic disease | very | Aspergillus sp. |
| Diarrhea, systemic disease | very | Aspergillus sp. |
| Diarrhea, thrush, vaginitis | very | Candida albicans |
| Systemic disease | very | Histoplasma capsulatum |
| Systemic disease | very | Coccidia |
| PARASITES | | |
| Trichomoniasis | very | Trichomonas |
| Diarrhea | very | Giardia |

What is claimed is:

1. A composition comprising transfer factor and an antibody, each independently derived from a source selected from the group consisting of ova, colostrum, blood, egg, and combinations thereof.

2. The composition of claim 1 wherein the transfer factor is lyophilized.

3. The composition of claim 1 wherein the antibody is lyophilized.

4. The composition of claim 1 wherein the source is bovine or avian.

5. The composition of claim 1 wherein the antibody is contained in an antibody fraction.

6. The composition of claim 5 wherein the antibody fraction is lyophilized.

7. The composition of claim 5 wherein the antibody fraction comprises antibodies at about 1% to about 99% per weight of the fraction.

8. The composition of claim 5 wherein the antibody fraction comprises antibodies at about 50% per weight of the fraction.

9. The composition of claim 1 wherein the transfer factor is encapsulated by a hydrophobic or lipid coating.

10. The composition of claim 1 wherein the antibody is encapsulated by a hydrophobic or lipid coating.

11. The composition of claim 5 wherein the antibody fraction is encapsulated by a hydrophobic or lipid coating.

12. The composition of claim 1 wherein both of the transfer factor and the antibody are encapsulated by a hydrophobic or lipid coating.

13. The composition of claim 5 wherein both of the transfer factor and the antibody fraction are encapsulated by a hydrophobic or lipid coating.

14. The composition of claim 5 wherein the antibody fraction is present from about 1% to about 99% by weight of the composition.

15. The composition of claim 5 wherein the antibody fraction is present at about 20% by weight of the composition.

16. The composition of claim 1 wherein the transfer factor is present from about 1% to about 99% by weight of the composition.

17. The composition of claim 1 wherein the transfer factor is present at about 80% by weight of the composition.

18. A composition comprising transfer factor, wherein the transfer factor is lyophilized.

19. A formulation comprising the composition of claim 1, 5, or 18 and a glucan.

20. The formulation of claim 19 wherein the glucan is hydrolyzed.

21. The formulation of claim 19 wherein the glucan is a fungal glucan.

22. The formulation of claim 21 wherein the fungus is a hybrid fungus.

23. The formulation of claim 19 wherein the glucan is supplied as a whole fungus organism.

24. The formulation of claim 23 wherein the fungus comprises a Cordyceps strain.

25. The formulation of claim 24 wherein the Cordyceps is Cordyceps sinensis.

26. The formulation of claim 23 wherein the glucan is present from about 10 mg to about 18 gm of whole organism per ounce.

27. The formulation of claim 23 wherein the glucan is present from about 100 mg to about 5 gm of whole organism per ounce.

28. The formulation of claim 19 wherein the glucan is encapsulated by a hydrophobic or lipid coating.

29. A formulation comprising the composition of claim 1, 5, or 18 wherein the formulation is encapsulated by a hydrophobic or lipid coating.

30. The formulation of claim 19 wherein the formulation is encapsulated by a hydrophobic or lipid coating.

31. The formulation of claim 19 further comprising a member selected from the group consisting of essential fats, lactic acid-producing bacteria, inositol hexaphosphate, olive leaf extract, aloe extract, β-sitosterol, yeast extract, montmorillonite, amino acids, methylsulfonylmethane, choline bitartrate, di-potassium phosphate, potassium chloride, magnesium sulfate, calcium pantothenate, vitamin E, vitamin C, vitamin A, vitamin D3, vitamin B1, vitamin B2, vitamin B12, zinc, and mixtures thereof.

32. The formulation of claim 19 further comprising a member selected from the group consisting of glucosamines, chondroitins, boswella, tumeric, super oxide dismutase, and mixtures thereof.

33. The composition of claim 1 or 18 wherein the transfer factor is a targeted transfer factor.

34. The composition of claim 33 wherein the targeted transfer factor is targeted to an organism selected from the group consisting of Herpes Simplex Virus 1, Herpes Simplex Virus 2, H. Pylori, Camphobactor, Chlamydia, Bovine Rhinotracheitis Virus, Parainfluenza, Respiratory Syncytial Virus Vaccine, modified live virus, Campylobacter Fetus, Leptospira Canicola, Grippotyphosa, Hardjo, Leterohaemorrhagiae, Pomona Bacterin, Bovine Rota-Coronaviras, Escherichia Coli Bacterin, Clostridium Chauvoei, Septicum, Haemolyticum, Novy, Sordellii, Perfringens Types C & D, Bacterin, Toxoid, Haemophilus Somnus, Pasteurella Haemolytica, Multocida Bacterin, and combinations thereof.

35. A kit comprising at least a first container comprising a composition comprising transfer factor, wherein said transfer factor is lyophilized.

36. A kit comprising at least a first container and a composition comprising transfer factor and an antibody.

37. The kit of claim 36 wherein the antibody is contained in an antibody fraction.

38. The kit of claim 36 wherein the transfer factor is lyophilized.

39. The kit of claim 36 wherein the antibody is lyophilized.

40. The kit of claim 37 wherein the antibody fraction is lyophilized.

41. The kit of claim 36 further comprising a second container comprising a reconstitution solution.

42. The kit of claim 36 further comprising instructions for reconstitution of one or more lyophilized components of the kit.

43. The kit of claim 36 further comprising instructions for administering the components to a subject.

44. A method of making a composition comprising the steps of:
A) Fractionating colostrum to obtain a first fraction having a molecular weight of about 10,000 Daltons or less;
B) Fractionating colostrum to obtain a second fraction having a molecular weight of about 10,000 to about 150,000 Daltons; and
C) Combining a first amount of the first fraction with a second amount of said second fraction to form the composition.

45. The method of claim 44 wherein the first amount contributes about 80% of the composition by weight, and the second amount contributes about 20% of the composition by weight.

46. The method of claim 44 additionally comprising the step of lyophilizing the first fraction.

47. The method of claim 44 additionally comprising the step of lyophilizing the second fraction.

48. The method of claim 44 additionally comprising the step of C) lyophilizing the composition.

49. A method of making a formulation comprising the steps of:
A) Fractionating colostrum to obtain a first fraction having a molecular weight of about 10,000 Daltons or less;
B) Fractionating colostrum to obtain a second fraction having a molecular weight of about 10,000 to about 150,000 Daltons;
C) Combining a first amount of the first fraction with a second amount of said second fraction to form a composition;
D) Combining the composition with a member selected from the group consisting of essential fats, lactic acid-producing bacteria, inositol hexaphosphate, olive leaf extract, aloe extract, β-sitosterol, yeast extract, montmorillonite, amino acids, methylsulfonylmethane, choline bitartrate, di-potassium phosphate, potassium chloride, magnesium sulfate, calcium pantothenate, vitamin E, vitamin C, vitamin A, vitamin D3, vitamin B1, vitamin B2, vitamin B12, zinc, and mixtures thereof.

50. A method of making a formulation comprising the steps of:
A) Fractionating colostrum to obtain a first fraction having a molecular weight of about 10,000 Daltons or less;
B) Fractionating colostrum to obtain a second fraction having a molecular weight of about 10,000 to about 150,000 Daltons;
C) Combining a first amount of the first fraction with a second amount of said second fraction to form a composition; and
D) Combining the composition with a member selected from the group consisting of glucosamines, chondroitins, boswella, turmeric, super oxide dismutase, and mixtures thereof.

51. A method of reducing tumor size in a subject in need thereof comprising administering to the subject a composition comprising transfer factor and an antibody or antibody fraction.

52. A method of reducing tumor size in a subject in need thereof comprising administering to the subject a composition comprising lyophilized transfer factor.

53. The method of claim 51 or 52 wherein the tumor size is reduced by at least about 50%.

54. The method of claim 51 or 52 wherein the tumor size is reduced by at least about 90%.

55. The method of claim 51 or 52 wherein the subject is avian or a mammal.

56. The method of claim 55 wherein the mammal is a human.

57. The method of claim 51 or 52 wherein the tumor is a malignant tumor.

58. The method of claim 57, wherein the malignant tumor is selected from the group consisting of a carcinoma, a melanoma, a sarcoma, a bone tumor, a mast cell tumor, a keratoma, a lymphoma, a histiocytoma, leukemia, and combinations thereof.

59. The method of claim 58, wherein the lymphoma is selected from the group consisting of Hodgkin's lymphoma and non-Hodgkin's lymphoma.

60. The method of claim 58 wherein the sarcoma is selected from the group consisting of a melanosarcoma, an osteosarcoma, and a hemangiosarcoma.

61. The method of claim 60, wherein the melanosarcoma is an amelanotic melanosarcoma.

62. The method of claim 58, wherein the carcinoma is an adenocarcinoma.

63. The method of claim 58, wherein the melanoma is an oral melanoma.

64. The method of claim 60, wherein the hemangiosarcoma is a splenic hemangiosarcoma.

65. A method of treating at least one condition in a subject comprising administering to the subject a composition comprising lyophilized transfer factor, wherein the condition is selected from the group consisting of hyperthyroidism, lymphopenia, Cushing's disease, Addison's disease, weight loss, hair loss, fatigue, and anorexia.

66. A method for the treatment of at least one condition in a subject comprising administering to the subject a composition comprising a transfer factor and an antibody or antibody fraction, wherein the condition is selected from the group consisting of hyperthyroidism, lymphopenia, Cushing's disease, Addison's disease, weight loss, hair loss, fatigue, and anorexia.

67. A method of reducing morbidity in a subject comprising the step of administering to the subject a composition comprising transfer factor and an antibody or antibody fraction.

68. A method of reducing morbidity in a subject comprising the step of administering to the subject a composition comprising lyophilized transfer factor.

69. A method of increasing feed conversion in a subject comprising the step of administering to the subject a composition comprising transfer factor and an antibody or antibody fraction.

70. A method of increasing feed conversion in a subject comprising the step of administering to the subject a composition comprising lyophilized transfer factor.
